(19) United States
(12) Patent Application Publication
(10) Pub. No.: US 2015/0088235 A1
(43) Pub. Date: Mar. 26, 2015

(54) DEVICE AND METHOD FOR WARMING AND STERILIZING SKIN FOR VEIN ACCESS

(71) Applicant: MARDA MEDICAL, INC., Indianapolis, IN (US)

(72) Inventors: David S. EVANS, Indianapolis, IN (US); Mark L. LOUGHERY, Indianapolis, IN (US); Greg OTTINGER, Noblesville, IN (US); Christopher D. BUCK, Brownsburg, IN (US); Philip L. RAMGE, Indianapolis, IN (US)

(21) Appl. No.: 14/496,283

(22) Filed: Sep. 25, 2014

Related U.S. Application Data
(60) Provisional application No. 61/911,873, filed on Dec. 4, 2013.
(60) Provisional application No. 61/882,365, filed on Sep. 25, 2013.

(51) Int. Cl. A61F 7/08

(52) U.S. Cl. CPC A61F 7/08
USPC 607/108

(57) ABSTRACT

A device and method are disclosed for improving vein access. An exemplary device includes a porous substrate saturated with a disinfectant. The device may maintain a temperature sufficient to cause vasodilation for an extended period of time after removal from a heat source.

### CROSS REFERENCE TO RELATED APPLICATIONS

This application claims priority to U.S. Provisional Patent Application Ser. No. 61/882,365, filed Sep. 25, 2013, and to U.S. Provisional Patent Application Ser. No. 61/911,873, filed Dec. 4, 2013, the disclosures of which are hereby expressly incorporated by reference herein in their entirety.

### FIELD OF THE DISCLOSURE

The present disclosure relates to vein access. More particularly, the present disclosure relates to a device for warming and sterilizing a patient's skin for vein access, and to a method for using the same.

### BACKGROUND AND SUMMARY

Today, there are over 170,000 medical facilities in the U.S. that need to access patients' veins on a daily basis. Vein access may be required to draw blood or to establish an IV line, for example.

It is often difficult to access a patient's vein on the first attempt, especially if the vein narrows and withdraws from the patient's skin surface through vasoconstriction. Vasoconstriction may be caused by low temperatures, fear, or shock and is prevalent among young and elderly patients. Requiring multiple needle sticks to access a constricted vein adds significant procedure time and cost, compounds patient distress, and increases the risk of infection.

Warming the intended puncture site may improve vein access through local vasodilation, which causes the underlying vein to dilate or “pop” and makes the vein easier to view and access. The approved clinical standard for blood collection suggests warming up to 107.6° F. (42° C.) “to dilate blood vessels and increase flow” (“Procedures for the Collection of Diagnostic Blood Specimens by Venipuncture; Approved Standard—Sixth Edition,” Clinical and Laboratory Standards Institute (CLSI), Vol. 27 (H3-A6), Section 7.13, 2007).

In current practice, a variety of different warming methods are used to warm the intended puncture site, including applying heated wash cloths, heated towels, or heat packs to the intended puncture site, for example. However, the intended puncture site must be cleaned after such warming steps, which may cool the patient's skin and counteract the benefit of any prior warming.

Another warming method is disclosed in U.S. Patent Application Publication No. 2007/0215634 to Levin, entitled “Individual Containers for Use in Medical Pad Warming Units,” which involves applying a heated, over-the-counter alcohol pad or cloth to the intended puncture site (See Paragraphs [0042]-[0043]). However, the present inventors have learned that such alcohol pads may lose heat and cool after about 5 seconds. By the time the alcohol pad is removed from the heat source, removed from its packaging, and applied to the patient's skin, the alcohol pad may be too cold to cause the desired vasodilation, and may actually cause undesired vasoconstriction as alcohol from the pad evaporates from and cools the patient's skin.

The present disclosure provides a device and method for improving vein access. An exemplary device includes a porous substrate saturated with a disinfectant. The device may maintain a temperature sufficient to cause vasodilation for an extended period of time after removal from a heat source.

According to an embodiment of the present disclosure, a device is provided for vein access including a porous substrate heated by an external heat source to at least a temperature sufficient to cause vasodilation at an intended puncture site, the substrate losing heat at a rate of about 2.0° C. or less per second when separated from the external heat source, and a disinfectant on the porous substrate.

According to another embodiment of the present disclosure, a device is provided for vein access including a foam substrate heated to at least a temperature sufficient to cause vasodilation at an intended puncture site, and a disinfectant on the foam substrate.

According to yet another embodiment of the present disclosure, a method is provided for vein access including the steps of heating a substrate with a heat source to at least a temperature sufficient to cause vasodilation, separating the substrate from the heat source, the substrate remaining at or above the temperature sufficient to cause vasodilation for at least about 7 seconds after the separating step, and applying the substrate to an intended puncture site.

### BRIEF DESCRIPTION OF THE DRAWINGS

The above-mentioned and other features and advantages of this disclosure, and the manner of attaining them, will become more apparent and the invention itself will be better understood by reference to the following description of embodiments of the invention taken in conjunction with the accompanying drawings, wherein:

FIG. 1 is a perspective view of an exemplary device for improving vein access, the device including a substrate that is saturated with a disinfectant;

FIG. 2 is a plan view of a package containing the substrate of FIG. 1, the package shown in a closed configuration;

FIG. 3 is a plan view of the package of FIG. 2 shown in an open configuration;

FIG. 4 is a flow chart illustrating an exemplary method for improving vein access using the substrate;

FIG. 5 is a graph illustrating the temperature of the substrate over time, the graph referencing the method steps of FIG. 4; and

FIG. 6 is a graph illustrating the temperature of the substrate over time in accordance with Example 2.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

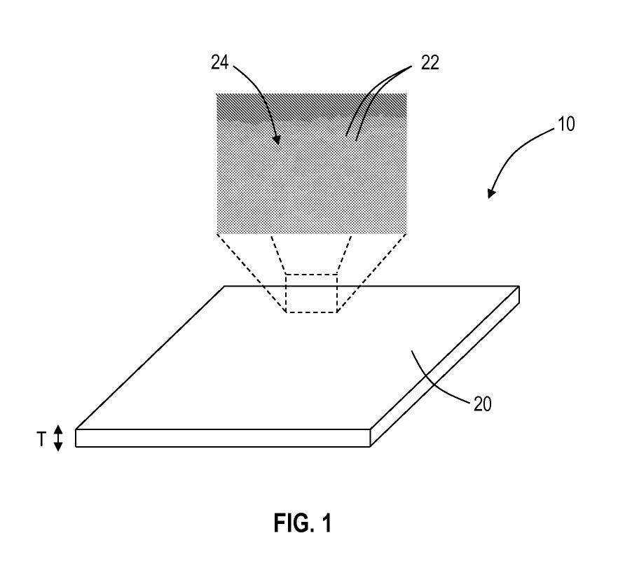

The present disclosure relates to a device and method for improving vein access. An exemplary device 10 is shown in FIG. 1 and includes a porous substrate 20 that is saturated with a disinfectant, such as alcohol (e.g., isopropyl alcohol), betadine, or another suitable disinfectant. The disinfectant may be in the form of a liquid, a cream, or a gel, for example.

The illustrative porous substrate 20 of FIG. 1 includes a plurality of intersecting frame members 22 that cooperate to define open cells or pores 24 therebetween. The frame members 22 may be constructed of suitable polymers or copolymers (e.g., polyethylene, polyurethane, ethylene vinyl acetate, etc.), natural or synthetic fibers (e.g., cotton), and/or optional composite fillers (e.g., calcium carbonate, carbon black, etc.). The open-cell nature of the substrate 20 may allow the disinfectant to distribute internally throughout the pores 24 of the substrate 20.

An exemplary porous substrate 20 is an open-cell polymer foam. The foam may be formed by trapping pockets of gas in a liquid or solid material, where the resulting material forms the frame members 22 and the pockets form the pores 24. Polyethylene foams, in particular, have been shown to withstand subsequent heating in the presence of alcohol without breaking down (See Example 1 below). In fact, polyethylene foams have been shown to exhibit increased tensile strength after heating. Exemplary foams include Capu-Cell™ Polyurethane Foam available from Time Release Sciences, Inc. of New York and P•E-Lite Ethylene Vinyl Acetate Copolymer Foam available from Inoac Corporation of Japan. It is also within the scope of the present disclosure that the substrate 20 may be formed by weaving or pressing together the frame members 22.

The frame members 22 that are exposed along the outer surfaces of the substrate 20 may give the substrate 20 an uneven, rough, or slightly abrasive texture. When the substrate 20 is applied to and moved across a patient's skin, the frame members 22 may create friction with the patient's skin 20 to produce a scrubbing effect. This friction may promote vasodilation and/or enhance bacteria destruction on the patient's skin. However, the frame members 22 may be sufficiently resilient to avoid scratching or breaking the patient's skin.

The substrate 20 may be more porous than a traditional over-the-counter alcohol pad. For example, the substrate 20 may have a porosity as low as about 40 ppi, 45 ppi, 50 ppi, 55 ppi, 60 ppi, 65 ppi, 70 ppi, 75 ppi, 80 ppi, 85 ppi, 90 ppi, 95 ppi, or 100 ppi and as high as about 110 ppi, 115 ppi, 120 ppi, 125 ppi, 130 ppi, 135 ppi, 140 ppi, 145 ppi, 150 ppi, or more, or within any range delimited by any pair of the foregoing values. For example, the substrate 20 may have a porosity between about 40 to 120 ppi, more specifically 50 to 85 ppi.

The substrate 20 may also be thicker than a traditional over-the-counter alcohol pad. For example, the substrate 20 may have a thickness (labeled “T” in FIG. 1) as low as about 1 mm, 2 mm, 3 mm, 4 mm, or 5 mm and as high as about 6 mm, 7 mm, 8 mm, 9 mm, 10 mm, or more, or within any range delimited by any pair of the foregoing values. This thickness may be measured before saturating the substrate 20 with the disinfectant and with the substrate 20 flat or unfolded. Applying the disinfectant to the substrate 20 may cause the substrate 20 to decrease in thickness. The traditional alcohol pad, by contrast, may have a thickness less than 1 mm when unfolded. The substrate 20 may be provided in the shape of a square, a rectangle, or any other suitable shape.

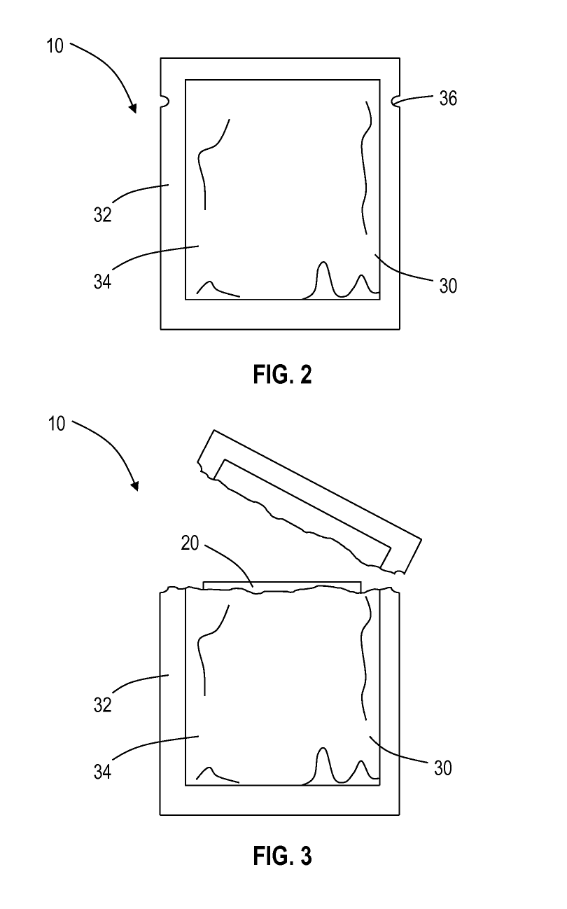

Each individual substrate 20 may be stored and heated in a sealed package 30, as shown in FIG. 2. The package 30 may include an outer sealed region 32, an inner region 34 sized to hold the substrate 20, and a separation mechanism 36 that facilitates access to the inner region 34 of the package 30. Exemplary separation mechanisms 36 include indentations, as shown in FIG. 2, perforations, and removable lids or covers, for example. The package 30 may be constructed of aluminum, plastic, or another suitable material. In the sealed or closed configuration of FIG. 2, this material may prevent the disinfectant on the substrate 20 from evaporating and prevent bacteria from entering the package 30. The package 30 may be torn or pulled open using the separation mechanism 36. In the open configuration of FIG. 3, the inner region 34 of the package 30 may be exposed to reveal the substrate 20 therein, and then the package 30 may be discarded.

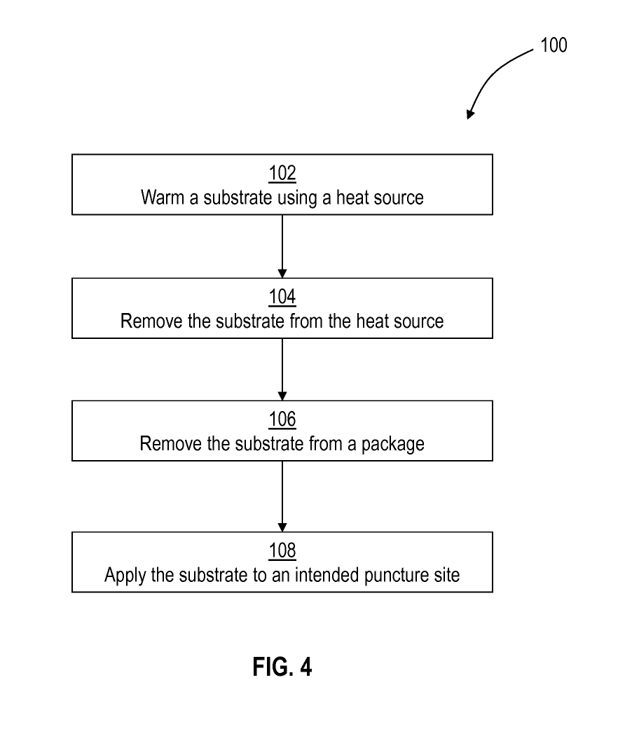

An exemplary method 100 for using the device 10 to improve vein access is shown in FIG. 4 and described below.

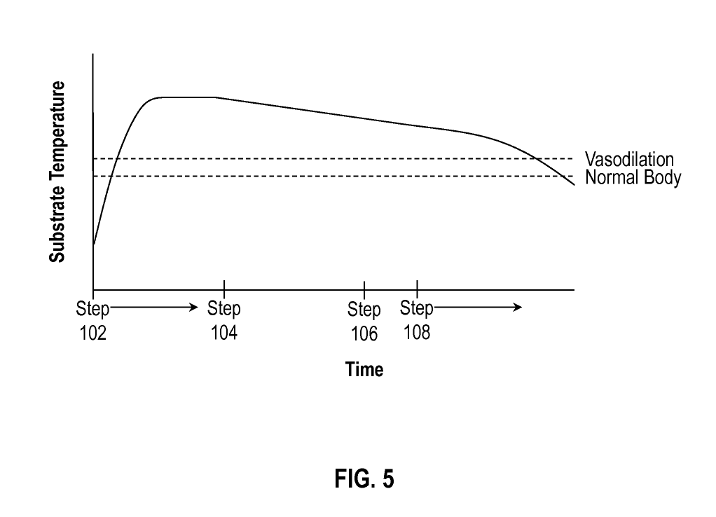

In step 102, the package 30 containing the substrate 20 may be warmed to at least a temperature sufficient to cause vasodilation at an intended puncture site using an external heat source. As used herein, the “temperature sufficient to cause vasodilation” or the “vasodilation temperature” means a temperature above the normal human body temperature of 98.6° F. (37° C.). Therefore, the substrate 20 may be warmed to a temperature above 98.6° F. (37° C.), such as about 104.0° F. (40° C.), 107.6° F. (42° C.), 111.2° F. (44° C.), 114.8° F. (46° C.), 118.4° F. (48° C.), 122.0° F. (50° C.), 125.6° F. (52° C.), 129.2° F. (54° C.), 131.0° F. (55° C.), 132.8° F. (56° C.), or more. The temperature of the substrate 20 should remain sufficiently low to be comfortable on the patient's skin and to avoid burning the patient's skin. Although the above-mentioned CLSI standard recommends warming temperatures up to 107.6° F. (42° C.), the present inventors have discovered that adult patients, in particular, may comfortably withstand even higher warming temperatures. Therefore, the substrate 20 may be warmed to a higher temperature for adult patients (e.g., 131.0° F. (55° C.)) than for young patients (e.g., 107.6° F. (42° C.)), for example. An exemplary graphical relationship between the normal body temperature, the vasodilation temperature, and the substrate temperature over time is shown in FIG. 5.

The warming step 102 may be performed while the package 30 is in the closed configuration of FIG. 3. The heat source may heat the substrate 20 and the package 30 by conduction or convection. The package 30 may be constructed of a material, such as aluminum, that facilitates heat transfer from the heat source to the substrate 20. Exemplary heat sources for use during the warming step 102 are disclosed in U.S. Pat. No. 6,316,750 to Levin, entitled “Apparatus for Warming Medical Pads,” and in U.S. Patent Application Publication No. 2007/0215634 to Levin, entitled “Individual Containers for Use in Medical Pad Warming Units,” the disclosures of which are expressly incorporated herein by reference in their entirety.

Returning to FIG. 4, the heated substrate 20 from the warming step 102 may then be separated from the heat source in step 104, removed from the package 30 in step 106, and applied to the patient's intended puncture site in step 108. The substrate 20 may be left unfolded inside the package 30, which may save time by avoiding the need to unfold the substrate 20 after the removing step 106. The applying step 108 may destroy bacteria at the intended puncture site to reduce the risk of infection during subsequent vein access. For example, performing the applying step 108 may decrease the log₁₀ microbial load count at the intended puncture site by about 1.0 CFU/cm², 1.5 CFU/cm², 2.0 CFU/cm², 2.5 CFU/cm², or 3.0 CFU/cm², or more. In certain embodiments, performing the applying step 108 for 15 seconds has been shown to decrease the log₁₀ microbial load count at the intended puncture site by 2.51 CFU/cm² to 2.73 CFU/cm². The duration of the applying step 108 may be as short as about 10 seconds, 15 seconds, or 20 seconds and as long as about 25 seconds, 30 seconds, or 35 seconds, or more, for example. The applying step 108 may end by removing the substrate 20 from the patient's skin, followed by accessing the patient's vein.

The substrate 20 begins to cool after the separating step 104 and continues to cool during the subsequent removing step 106 and applying step 108, as shown in FIG. 5. The cooling pattern may vary depending on the duration of the removing step 106 and the applying step 108 and the time that passes between each step. For example, the longer the substrate 20 remains inside the package 30 before the removing step 106, the more heat the substrate 20 may retain. Also, the sooner the substrate 20 is applied to the patient's skin rather than being surrounded by room temperature air, the more heat the substrate 20 may retain. Advantageously, the porosity and/or thickness of the substrate 20 may allow the substrate 20 to exhibit better heat retention than a traditional over-the-counter alcohol pad.

In certain embodiments, the substrate 20 may maintain at least the vasodilation temperature (i.e., remain at or above the vasodilation temperature) for a longer period of time than a traditional over-the-counter alcohol pad. For example, after the separating step 104, the substrate 20 may maintain at least the vasodilation temperature for a time as short as about 7 seconds, 8 seconds, 9 seconds, 10 seconds, or 15 seconds, and as long as about 20 seconds, 30 seconds, 40 seconds, 50 seconds, 60 seconds, 70 seconds, or more, or within any range delimited by any pair of the foregoing values.

In other embodiments, the substrate 20 may lose heat at a slower rate than a traditional over-the-counter alcohol pad. For example, after the separating step 104, the substrate 20 may lose heat at a rate as high as about 2.0° C., 1.5° C., 1.0° C., or 0.5° C. per second, and as low as about 0.30° C., 0.25° C., 0.20° C., 0.15° C., 0.10° C., 0.05° C., or less per second, or within any range delimited by any pair of the foregoing values.

According to an exemplary embodiment of the present disclosure, the heat retention of the substrate 20 may allow the removing step 106 and the applying step 108 to be completed with the substrate 20 at or above the vasodilation temperature, as shown in FIG. 5. For example, if a clinician performs the removing step 106 and the applying step 108 in about 10 to 15 seconds after the separating step 104, the substrate 20 may stay at or above the vasodilation temperature during those 10 to 15 seconds. This heat retention may promote vasodilation and avoid vasoconstriction at the intended puncture site during the applying step 108. Also, this heat retention may enhance bacteria destruction by the disinfectant at the intended puncture site during the applying step 108 to reduce the risk of infection.

The device 10 may be sterilized before use. For example, the substrate 20 and/or the package 30 may be exposed to sterilizing radiation or chemicals before use. The sterile condition of the device 10 may improve the shelf-life of the device 10. For example, the device 10 may have a shelf-life of about 6 months, 9 months, 1 year, or more. Also, the sterile condition of the device 10 may improve bacteria destruction at the intended puncture site during the applying step 108. Furthermore, the sterile condition of the device 10 may allow the clinician to avoid any subsequent cleaning steps after the applying step 108. Instead, the clinician may access the patient's vein immediately after the applying step 108.

### EXAMPLES

The following examples are to be considered as illustrative in nature, and are not limiting in any way.

### 1. Stability of Heated Substrates

First samples of the above-described Capu-Cell™ Polyurethane Foam and second samples of the above-described P•E-Lite Ethylene Vinyl Acetate Copolymer Foam were saturated with alcohol and exposed to elevated temperatures of about 131.0° F. (55° C.) over time. At specific time intervals up to a maximum time interval of 6 weeks (which simulates about 18 months of accelerated aging), the foam samples were subjected to tensile strength testing and elongation testing. The first, polyurethane foam samples exhibited significant mechanical degradation and broke down after 6 weeks of heating. However, the second, polyethylene foam samples did not exhibit significant mechanical degradation, even after the maximum 6 weeks of heating. In fact, the second, polyethylene foam samples exhibited increased tensile strength after 6 weeks of heating.

### 2. Heat Retention by Substrates

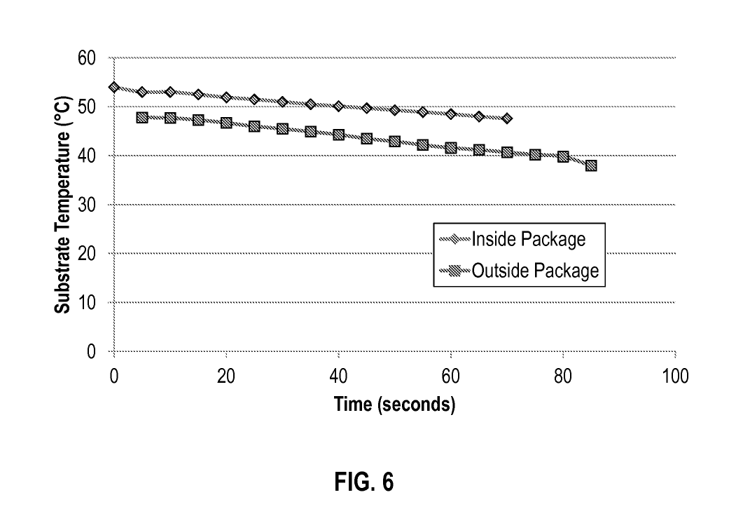

Packaged substrates were heated to elevated temperatures of about 122.0° F. (50° C.) using an external heat source. The packages were then removed from the heat source (time=0). Some of the substrates were then removed from the packages and placed on a patient's skin, and other substrates were left inside the packages. The temperature of each substrate was monitored over time. As shown in FIG. 6, the substrates cooled gradually over time, remaining above 98.6° F. (37° C.) for more than 70 seconds. The substrates that were left inside the packages exhibited better heat retention than the substrates that were removed from the packages. Specifically, the substrates that were left inside the packages lost heat at a rate of about 0.09° C. per second, while the substrates that were removed from the packages lost heat at a higher rate of about 0.12° C. per second.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A device for vein access comprising:
a porous substrate heated by an external heat source to at least a temperature sufficient to cause vasodilation at an intended puncture site, the substrate losing heat at a rate of about 2.0° C. or less per second when separated from the external heat source; and
a disinfectant on the porous substrate.

2. The device of claim 1, wherein the porous substrate loses heat at a rate of about 0.3° C. or less per second when separated from the external heat source.

3. The device of claim 2, wherein the porous substrate loses heat at a rate of about 0.15° C. or less per second when separated from the external heat source.

4. The device of claim 1, wherein the porous substrate includes a plurality of frame members that define open pores therebetween.

5. The device of claim 1, wherein the porous substrate comprises at least one of polyethylene and polyurethane.

6. The device of claim 1, wherein the porous substrate has a thickness of 1 mm or more.

7. A device for vein access comprising:
a foam substrate heated to at least a temperature sufficient to cause vasodilation at an intended puncture site; and
a disinfectant on the foam substrate.

8. The device of claim 7, wherein the foam substrate has a porosity of about 40 to 120 ppi.

9. The device of claim 8, wherein the foam substrate has a porosity of about 50 to 85 ppi.

10. The device of claim 7, wherein the foam substrate has a thickness of 1 mm or more.

11. The device of claim 10, wherein the foam substrate has a thickness of about 4 mm or more.

12. The device of claim 7, wherein the foam substrate comprises at least one of polyethylene and polyurethane.

13. A method for vein access comprising the steps of:
heating a substrate with a heat source to at least a temperature sufficient to cause vasodilation;
separating the substrate from the heat source, the substrate remaining at or above the temperature sufficient to cause vasodilation for at least about 7 seconds after the separating step; and
applying the substrate to an intended puncture site.

14. The method of claim 13, wherein the temperature sufficient to cause vasodilation exceeds 37° C.

15. The method of claim 13, wherein the heating step comprises heating the substrate to a temperature of about 42° C.

16. The method of claim 13, wherein the heating step comprises heating the substrate to a temperature of about 55° C.

17. The method of claim 13, wherein the substrate remains at or above the temperature sufficient to cause vasodilation for at least about 10 seconds after the separating step.

18. The method of claim 17, wherein the substrate remains at or above the temperature sufficient to cause vasodilation for at least about 20 seconds after the separating step.

19. The method of claim 13, wherein the applying step occurs while the substrate remains at or above the temperature sufficient to cause vasodilation.

20. The method of claim 13, further comprising the step of removing the substrate from a package, wherein the removing step occurs while the substrate remains at or above the temperature sufficient to cause vasodilation.
